(19) United States
(12) Patent Application Publication
(10) Pub. No.: US 2019/0221310 A1
(43) Pub. Date: Jul. 18, 2019

(54) SYSTEM AND METHOD FOR AUTOMATED DIAGNOSIS AND TREATMENT

(71) Applicant: James Stewart Bates, Paradise Valley, AZ (US)

(72) Inventor: James Stewart Bates, Paradise Valley, AZ (US)

(21) Appl. No.: 15/872,274

(22) Filed: Jan. 16, 2018

(51) Int. Cl. G16H 50/20; G16H 10/60; G16H 15/00; G16H 70/60; G16H 30/40; G16H 70/20; G16H 50/30

(52) U.S. Cl. CPC G16H 50/20; G16H 10/60; G16H 15/00; G16H 50/30; G16H 30/40; G16H 70/20; G16H 70/60

(57) ABSTRACT

Various embodiments of the present application relate to systems and methods for automated diagnosis and treatment, especially for automated diagnosis and treatment integrated with artificial intelligence as well as incorporating treatment scheme feedback. The automated healthcare diagnosis and treatment comprises a doctor apparatus to receive patient symptom input, electronic history records, retrieved reference information and diagnostic results presented by a diagnostic engine. The doctor may choose one presented diagnostic result as the identified diagnostic result or request further diagnostic result for further reference. Furthermore, the doctor may follow generated treatment schemes based on the identified diagnostic result, or input a new treatment scheme different from the generated treatment schemes. The decision of the doctor is used as a feedback to the system for diagnostic and treatment module update. The application of these methods may result in an improvement in efficiency and consistency for automated diagnosis and treatment.

### BACKGROUND

### A. Technical Field

The present application relates generally to systems and methods for automatic diagnosis and treatment services, especially for automated healthcare diagnosis and treatment capable of automatically retrieve reference and generating diagnostic information.

### B. Background of the Invention

With the healthcare industry's continuous driven for improving efficiency and throughput, automation of healthcare services becomes an important part of a strategy for performance improvement. Automation involves the application of control systems and information technologies to reduce the need for human work in the production of goods and services. Benefits provided by automated healthcare services include but not limited to labor savings, improved consistency, increased predictability, and high throughput, etc. Furthermore, automated healthcare services may generate lots of data which can be used for continuous performance optimization and improvement.

Among automated healthcare services, healthcare services integrated with artificial intelligence (AI) have become more and more popular. In healthcare application, AI uses various algorithms and software to approximate human cognition in the analysis of complex medical data to incrementally improve patient medical records, care delivery, diagnostic accuracy, and drug development, etc.

The implementation of AI not only improves care delivery, but also assists in clinician decision-making and operational efficiency, amplifying the impact of each individual practitioner. Although rapid progress has been made, various challenges still exist in implementing in automated healthcare, such as the requiring of robust dataset with both the breadth and depth for training in a particular application, patient health record recognition, and translation, incorporating latest medical developments, challenges in natural language processing, rare disease detection, seamlessly updating the automated healthcare diagnosis and treatment algorithms, etc.

What are needed are systems, devices and methods for automated healthcare services integrated with AI that can be smoothly implemented in diagnosis and treatment for clinician practices and patient care.

### BRIEF DESCRIPTION OF THE DRAWINGS

Reference will be made to exemplary embodiments of the present application that are illustrated in the accompanying figures. Those figures are intended to be illustrative, rather than limiting. Although the present application is generally described in the context of those embodiments, it is not intended by so doing to limit the scope of the present application to the particular features of the embodiments depicted and described.

FIG. 1 shows an exemplary automated healthcare system in accordance with various embodiments of the present application.

FIG. 2 shows an exemplary component diagram of a healthcare provider (e.g. doctor) apparatus in accordance with various embodiments of the present application.

FIG. 3 shows an exemplary modular diagram of an automated healthcare system in accordance with various embodiments of the present application.

FIG. 4 is an exemplary flow diagram illustrating a process of doctor user authentication according to various embodiments of the present application.

FIG. 5 is an exemplary flow diagram illustrating a process for diagnostic and treatment according to various embodiments of the present application.

FIG. 6 is another exemplary flow diagram illustrating a process for diagnostic and treatment according to various embodiments of the present application.

FIG. 7 is another exemplary flow diagram illustrating a process for diagnostic and treatment according to various embodiments of the present application.

FIG. 8 is an exemplary flow diagram illustrating a process of billing according to various embodiments of the present application.

FIG. 9 is a simplified block diagram of a computing device/information handling system according to various embodiments of the present disclosure.

One skilled in the art will recognize that various implementations and embodiments of the present application may be practiced in accordance with the specification. All of these implementations and embodiments are intended to be included within the scope of the present application.

### DETAILED DESCRIPTION OF THE PREFERRED EMBODIMENTS

In the following description, for purpose of explanation, specific details are set forth in order to provide an understanding of the present application. The present application may, however, be practiced without some or all of these details. The embodiments of the present application described below may be incorporated into a number of different electrical components, circuits, devices, and systems. Structures and devices shown in block diagram are illustrative of exemplary embodiments of the present application and are not to be used as a pretext by which to obscure broad teachings of the present application. Connections between components within the figures are not intended to be limited to direct connections. Rather, connections between components may be modified, re-formatted, or otherwise changed by intermediary components. Furthermore, one skilled in the art will recognize that embodiments of the present invention, described below, may be implemented in a variety of ways, such as a process, an apparatus, a system, a device, or a method on a tangible computer-readable medium.

Components, or modules, shown in diagrams are illustrative of exemplary embodiments of the invention and are meant to avoid obscuring the invention. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components. Components may be implemented in software, hardware, or a combination thereof.

Furthermore, connections between components or systems within the figures are not intended to be limited to direct connections. Rather, data between these components may be modified, re-formatted, or otherwise changed by intermediary components. Also, additional or fewer connections may be used. It shall also be noted that the terms “coupled,” “connected,” or “communicatively coupled” shall be understood to include direct connections, indirect connections through one or more intermediary devices, and wireless connections.

Reference in the specification to “one embodiment,” “preferred embodiment,” “an embodiment,” or “embodiments” means that a particular feature, structure, characteristic, or function described in connection with the embodiment is included in at least one embodiment of the invention and may be in more than one embodiment. Also, the appearances of the above-noted phrases in various places in the specification are not necessarily all referring to the same embodiment or embodiments.

The use of certain terms in various places in the specification is for illustration and should not be construed as limiting. A service, function, or resource is not limited to a single service, function, or resource; usage of these terms may refer to a grouping of related services, functions, or resources, which may be distributed or aggregated.

The terms “include,” “including,” “comprise,” and “comprising” shall be understood to be open terms and any lists the follow are examples and not meant to be limited to the listed items. Any headings used herein are for organizational purposes only and shall not be used to limit the scope of the description or the claims. Each reference mentioned in this patent document is incorporate by reference herein in its entirety.

Furthermore, one skilled in the art shall recognize that: (1) certain steps may optionally be performed; (2) steps may not be limited to the specific order set forth herein; (3) certain steps may be performed in different orders; and (4) certain steps may be done concurrently.

According to various embodiments, systems and methods for automated diagnosis and treatment are presented. In some embodiments, an automated healthcare system comprises a patient apparatus, a doctor apparatus, at least one self-measurement apparatus, a patient management server, computing apparatus, and a storage storing electronic medical record database. Those apparatuses are coupled wired or wirelessly via one or more networks for information exchange. Each of the apparatuses may be configured to comprise various functional components. Alternatively, some of the apparatuses may be integrated together as single equipment. Application software or APP may be installed on those apparatus to implement desired functionalities. One skilled in the art will recognize that these structural and functional components, described below, may be combined in various manners across embodiments of the present application.

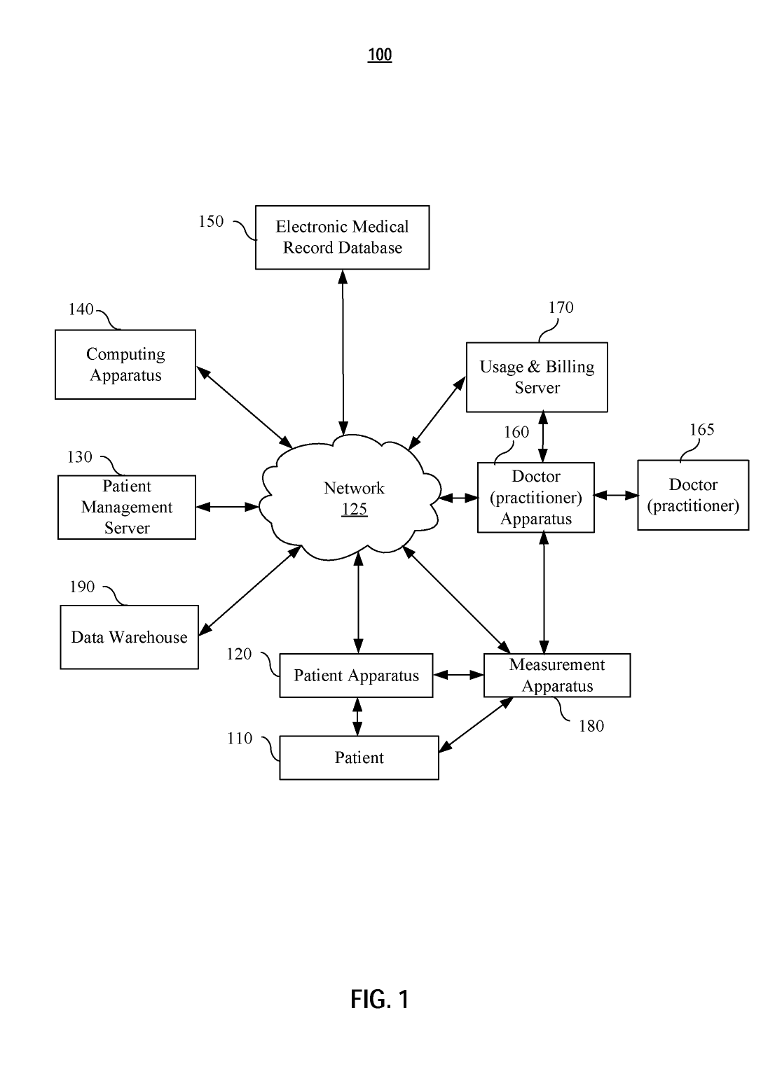

Embodiments of the present application will now be described in reference to FIGS. 1-9. Thereafter, specific embodiments of an automated healthcare service will be described with various structural and functional components. One skilled in the art will understand these embodiments may be implemented at various environments including, but not limited to, the hospital, senior care facility, nursing home, and recovery center, etc.

FIG. 1 shows an exemplary automated healthcare system in accordance with various embodiments of the present application. The automated healthcare system 100 comprises a patient apparatus 120, a patient/doctor management server 130, a computing apparatus 140, an electronic medical record database (such as internal EHR database or external EHR database) 150, a doctor apparatus 160, a usage/billing server 170, at least one measurement apparatus 180 (which may be a self-measurement apparatus), and one or more data warehouse 190 that contains symptom, illness, treatment outcome, coding relationships, or meta data used for diagnostic, treatment and coding related algorithms. Those apparatuses are coupled in wired or wireless links via one or more networks 125 for information exchange. Each of the apparatuses may be configured to comprise various functional components. Alternatively, some of the apparatuses may be integrated together as single equipment. Application software or APP may be installed on those apparatus to implement desired functionalities.

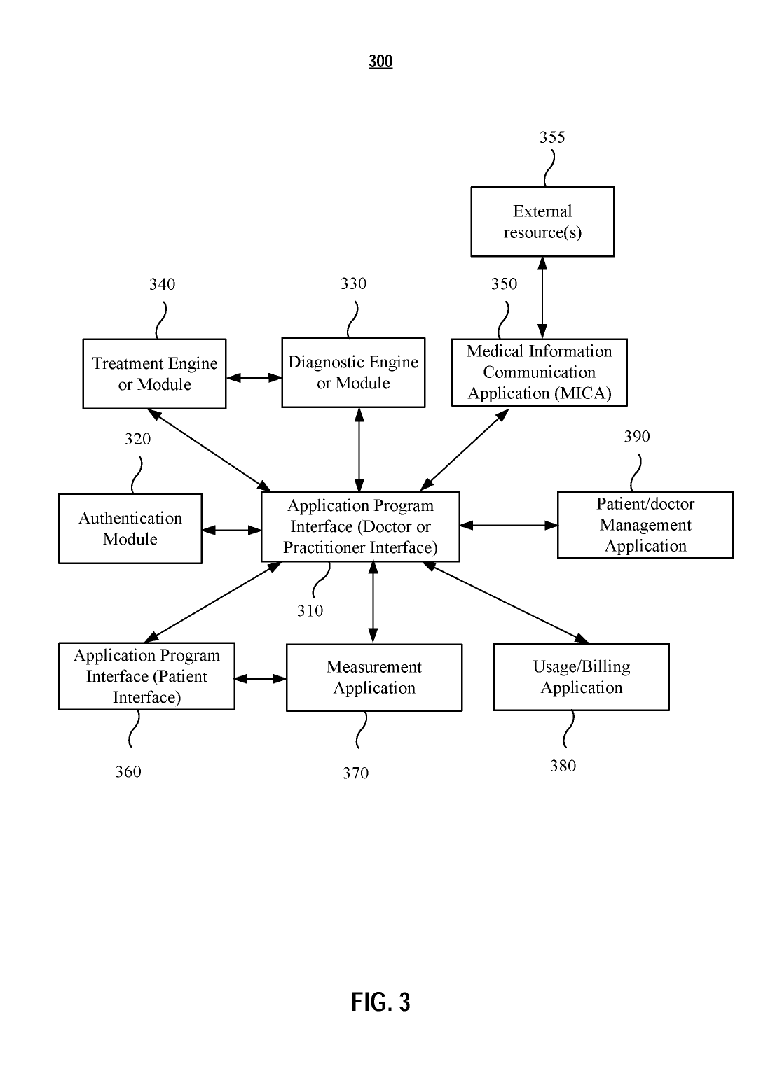

Both the patient apparatus 120 and the doctor apparatus 160 (also referred as practitioner apparatus hereinafter) may be a tablet, computer, mobile device, or other electronic device, having an application program interface (API) for user interaction. The API may be a doctor interface 310 (as shown in FIG. 3, also referred as practitioner interface hereinafter), a patient interface 360 (shown in FIG. 3), or a graphic user interface (GUI) configurable as various screen views to users (e.g., patients, doctors/nurses, operation managers, and administrators) based on user authorization level and other parameters. The API may interact with other components (e.g., touch screen, keyboard, and sensors) within the apparatus, and/or even other apparatus (e.g. the computing apparatus 140 and the patient/doctor management server 130, etc.). The API may provide audio/visual instructions on measurements (e.g., animation view and real time stream of the user).

In some embodiments, the patient apparatus 120 is a smart phone with a mobile app installed. The mobile app may be provided by the healthcare provider (such as a hospital) via a downloadable link. Once the patient registers or checked in, the downloadable link is sent to the patient's mobile phone. Afterwards, the patient is able to download, install and follow the instruction from the mobile app. In some embodiments, the patient apparatus 120 is a patient kiosk, which is installed at facility of the healthcare provider to provide self-guided service for the patient user. The Kiosk may integrate one or more self-management apparatus 180 for additional functionalities.

In operation, upon registration and authentication, a patient 110 may enter symptom-related data, such as type and duration of symptoms, health concerns, medical instrument measured diagnostic data, images, sound patterns, or other relevant information into the patient apparatus 120. The patient may use various ways of communication, such as voice control, to enter data, e.g., in the form of a machine-driven questionnaire. The patient apparatus 120 may communicate the data raw or in processed form with other apparatus via the network 125 in wired or wireless communication. The network 125 may be a local area network (LAN), a wide area network (WAN), a storage area network (SAN), or a combination thereof. The network 125 may be configured to support various communication methods, including but not limited to Wi-Fi, Ethernet, Bluetooth, optical fiber, etc.

In embodiments, the patient apparatus 120 may transmit the data in an encrypted format to other apparatuses via the network 125. The computing apparatus 140 may generate further request/question or diagnostic information based on the data received from the patient apparatus 120, and the one or more data warehouse 190 that contains symptom, illness, treatment outcome, coding relationships, etc. The generation of further request/question or diagnostic information may be implemented using an artificial intelligence (AI) based process. The AI based process may involve software and/or hardware having a program of instructions embodied thereon, or a combination thereof. The generated request and/or question are transmitted back to the patient apparatus 120 for the patient to input more data accordingly. The generated diagnostic information is transmitted back to the doctor apparatus 160 for reference. In some embodiments, the computing apparatus 140 also search for a reference from the electronic medical record database 150 based on the data received from the patient apparatus 120 and the one or more data warehouse 190 that contains symptom, illness, treatment outcome, coding relationships, etc. The reference may be health history of the patient, allergy history, background medical information related to the patient's input, common diseases related to patient's symptoms, common treatments related to patient's symptoms, latest medical discovery related to the patient's input, etc. In some embodiments, the reference is also generated/retrieved using an AI based process. The AI process for the reference generation and the AI process for the generation of further request/question or diagnostic information may share same hardware, but use separate software modules. The computing apparatus 140 may be a centralized server or a cloud based server loaded with machine-executable instructions that may be in program modules that are executed by a processing device.

In embodiments, the patient/doctor management server 130 and usage/billing server 170 receive data from the patient apparatus 120 and/or the computing apparatus 140, such that the patient history can be updated and patient billing information may be generated accordingly. In embodiments, the computing apparatus 140 and the usage/billing server 170 also couples to the doctor apparatus 160, such that the doctor can access the information provided by these apparatuses. Furthermore, the time and cession usage from the doctor may also be tracked for record or billing purpose.

In embodiments, the computing apparatus 140, the patient management server/doctor 130 and usage/billing server 170 may be integrated as a single server to implement function of all three apparatuses. Furthermore, the electronic medical record database 150 and the one or more data warehouse 190 that contains symptom, illness, treatment outcome, coding relationships, etc., may be stored within a memory of the single server. In other embodiments, the electronic medical record database 150 may be a third-party database accessible by the single server.

In embodiments, at least one measurement apparatus 180 couples to the patient apparatus 120 and/or the doctor apparatus 160. The patient is prompted via the patient apparatus 120 to use the measurement apparatus 180 for some medical self-measurement in an effort to aid medical diagnosis. The self-measurement may be requested by a doctor, recommended by the computing apparatus 140 based on patient's input information, or by a routine check procedure. A software application may be implemented on the patient apparatus 120 to provide guidance to use the measurement apparatus 180. In some embodiments, various processes, including process with augmented reality, may be implemented to ensure that the measurement apparatus 180 is properly used. Gathered measurement data may be transmitted from the patient apparatus 120 and/or the measurement apparatus 180 to the computing apparatus 140 and the doctor apparatus 160 as reference for further reaction.

The measurement apparatus 180 is capable of measuring one or more characteristics of a patient. In embodiments, the measurement apparatus 180 is a combination of diagnostic medical devices that generate diagnostic data based on patient characteristics. Exemplary self-measurement apparatus may be heart rate sensors, otoscopes, digital stethoscopes, in-ear thermometers, blood oxygen sensors, high-definition cameras, spirometers, blood pressure meters, respiration sensors, skin resistance sensors, glucometers, ultrasound devices, electrocardiographic sensors, body fluid sample collectors, eye slit lamps, weight scales, and any devices known in the art that may be operated by the patient in performing a medical diagnosis. In embodiments, the patient apparatus 120 is preload with necessary application software such that the patient just plug-in and use the self-measurement apparatus 180. Alternatively, the measurement apparatus 180 may be a standalone apparatus for the patient to use. The gathered measurement data may be transmitted directly from the measurement apparatus 180, or via the patient apparatus 120, to other apparatuses, such as the computing apparatus 140 or the doctor apparatus 160.

In embodiments, the measurement apparatus 180 also comprises camera(s), proximity sensor, or other components to track the movement of the patient. Further, algorithms with augmented reality (AR) may be implemented to guide the patient to move the sensor to a desired target location for accurate and reliable measurements. Once the sensor, e.g., a stethoscope is placed at the desired target location on a patient's torso, the patient may be prompted by optical or visual cues shown on screen on the patient apparatus 120 or the measurement apparatus 180 to breathe according to instructions or perform other actions to facilitate medical measurements and to start a measurement.

In embodiments, a doctor (or healthcare provider) 165 and a patient 110 are paired based on a pairing process. The pairing process may be a pre-determined process or a dynamically determined process. For example, the pairing process may be based on appointment, patient location, symptom, doctor's specialty, doctor's availability, cost standard, etc. In embodiments, the doctor 165 and the patient 110 (or the patient apparatus 120 and the doctor apparatus 160) are paired before the automated diagnostic started. In embodiments, the doctor 165 and the patient 110 (or the patient apparatus 120 and the doctor apparatus 160) are paired after an automated diagnostic module presents one or more diagnostic results. The pairing process may be initiated by the patient or by the automated healthcare system.

In embodiments, the patient apparatus 120 and the doctor apparatus 160 are not located in the same facility. For example, a remotely located doctor may log in the automatic healthcare system 100 via the doctor apparatus 160 and provide instructions to the patient, e.g., by communicating via the network 125. Diagnostic results and treatment suggestions provided by the automated healthcare system 100 are accessible by the doctor for review, confirmation, or even modification if the doctor has different opinion. Input from the doctor may be transmitted back for further reaction, including treatment, billing, drug prescription, patient history update, etc.

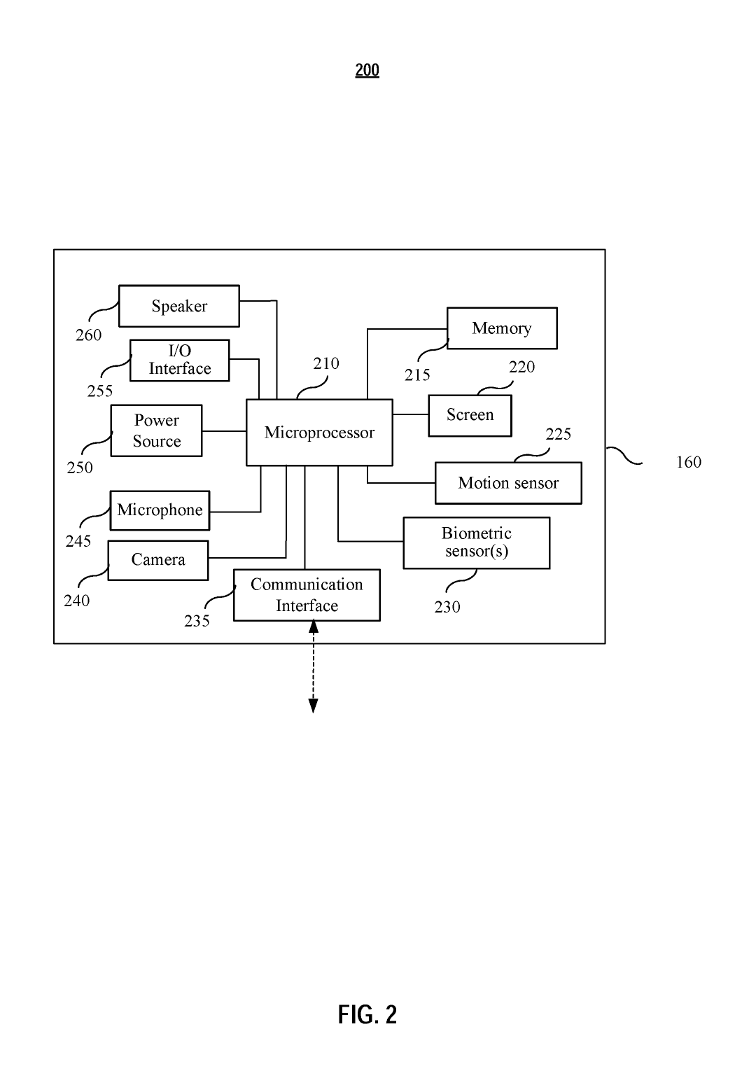

FIG. 2 shows an exemplary component diagram of a doctor apparatus in accordance with various embodiments of the present application. As depicted, the doctor apparatus 160 comprises microprocessor 210, memory 215, screen 220, motion sensor 225, biometric sensor(s) 230, communication interface 235, camera 240, microphone 245, power source 250, I/O interface 255, and speaker 260. The patient apparatus 120 may further comprise additional components, may integrated with the one or more self-measurement apparatus. Some of the aforementioned components may be integrated into a single component. For example, the screen 220 and the I/O interface 255 may be integrated together into a touch screen.

In embodiments, the doctor apparatus 160 may securely receive information in encrypted form to ensure privacy and the authenticity of patient input, as well as gathered measurement data from the measurement apparatus 180. Such encrypted communication may be accomplished by applying of security features embedded in hardware of the doctor apparatus 160 and/or software that enable security features during transit and storage of sensitive data.

In embodiments, the communication interface 235 is an interface to enable bi-directional wired or wireless communications. For example, the communication interface 235 may transmit processed or raw data using various wireless communication protocol known in the art, such as Wi-Fi, Bluetooth, etc. For example, the communication interface 235 may receive instructions, in a text, audio or video format, for the patient to respond.

In embodiments, the doctor apparatus 160 comprises at least one biometric sensor 230 to collect biometric information from the doctor for doctor identification and/or authentication. The biometric sensor 230 may be a fingerprint sensor, an iris scanner, a DNA, etc. In some embodiment, the biometric sensor 230 is a camera or microphone. The doctor apparatus 160 utilizes pre-loaded software to implement facial or voice recognition through the camera or microphone for identification and/or authentication.

In some embodiments, the doctor apparatus 160 is a smart phone, a tablet, or a notebook, etc., with desired automated healthcare service app installed. The app may be developed or provided by a third party (such as a medical resource management company) via a downloadable link, which is provided to the doctor upon doctor registration. After installation, an application program interface (API) is rendered on the doctor apparatus 160 with instructions to guide the doctor to use the automated healthcare service for diagnosis and treatment.

One of ordinary skill in the art shall understand that the aforementioned exemplary component diagram of a doctor apparatus 160 may also be applicable to the patient apparatus 120.

FIG. 3 shows an exemplary modular diagram 300 of an automated healthcare system in accordance with various embodiments of the present application. The application program interface (API) 310 displayed on the screen of the doctor apparatus 160 is a specially designed interface portal that brings information from diverse sources together in a coherent and consistent way presentable to the doctor. The diverse sources may be an authentication module 320, a diagnostic engine (or diagnostic module) 330, a treatment engine (or treatment module) 340, a medical information communication application (MICA) module 350 coupled to one or more external resources 355, one or more measurement application 370 (which could be a self-measurement application), and usage/billing application 380, etc. In embodiment, both the treatment engine 340 and the diagnostic engine 330 are coupled to the doctor interface 310 in a doctor apparatus 160 (not shown in FIG. 3). The modules, engines, or applications shown in FIG. 3 may be referred as software and/or hardware having a program of instructions embodied thereon, or a combination thereof.

In embodiments, the modules, engines, or applications shown in FIG. 3 are implementable at different apparatuses coupled to each other via the network 125 (not shown in FIG. 3). In embodiments, some of the modules, engines, or applications are implemented at the same apparatus. For example, the API 360 and the measurement application 370 may both be installed at the patient apparatus 120 (e.g. patient Kiosk). The diagnostic engine 330, the treatment engine 340, and the MICA module 350, may all be installed at the computing apparatus 140. The usage/billing application 380 may be installed together with the patient/doctor management application 390 in a hospital server.

In embodiments, the API 310 and API 360 may further couple to an internal Electronic Health Record (EHR) database, which may be integrated within the patient/doctor management application 390. Input from the patient through the API 360, such as newly reported symptoms, updated drug history, etc., may be used to update the patient's internal EHR. The internal EHR is an electronic version of a patient's medical history, that is maintained and/or updated by the healthcare provider over time, and may include administrative clinical data relevant to the patient under the healthcare provider, including demographics, progress notes, problems, medications, vital signs, past medical history, immunizations, laboratory data and radiology reports. The internal EHR provides benefits to streamline the clinician's workflow. In embodiments, the internal EHR also has the ability to support other care-related activities directly or indirectly through various interfaces, including evidence-based decision support, quality management, and outcomes reporting. EHRs are important in the continued progress of healthcare that can strengthen the relationship between patients and healthcare providers. The data, and the timeliness and availability of it, would enable providers to make better decisions and provide better care. A well-documented EHR can improve patient care by reducing the incidence of medical error by improving the accuracy and clarity of medical records, by making the health information available, reducing duplication of tests, reducing delays in treatment, and patients well informed to take better decisions, and/or reducing medical error by improving the accuracy and clarity of medical records.

In embodiments, the API 310 (as well as the API 360) may further couple to MICA module 350 such that a doctor (or the patient) may request/fetch reference information from external resource(s) 355. MICA may be populated by natural language processing of outside data sources to extract the symptom, illness, treatment, outcome results. The reference information may be external EHR of the patient, medical knowledge related to symptom input by the patient, reports on related symptoms, illness, or treatment, etc. The medical knowledge may include possible diagnosis and treatment solutions. The external resource(s) 355 may comprise external EHR database maintained by third-party healthcare providers, medical insurance database maintained by medical insurance company, medical knowledge database such as PubMed or Medline, etc. In embodiments, the possible diagnosis results and treatment solutions are ranked and only certain top diagnosis results and treatment solutions are transmitted back for the doctor's reference.

In embodiments, the reference information may also include information extracted from non-medical related sources, such as the social media, web post, etc. The reference information may include but not limit to life style information or analysis based on the patient's post on various social media. That reference information may be used by the diagnostic engine 330 for credibility check of the patient's input, and assistance in diagnostics, etc.

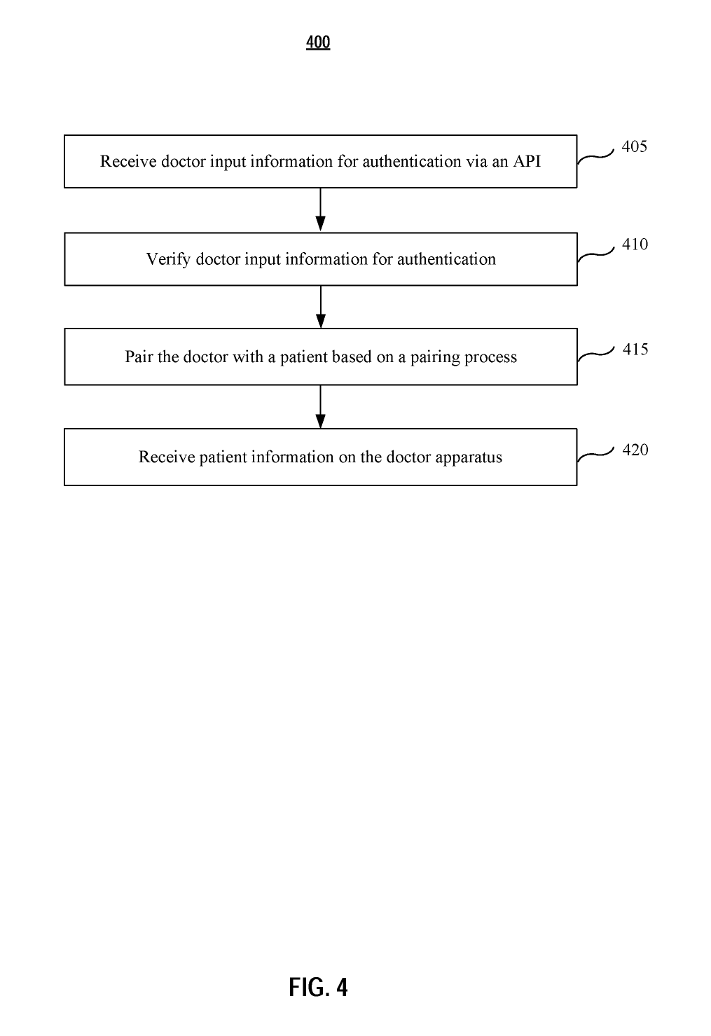

In embodiments, the diagnostic engine 330, the treatment engine 340, the MICA module 350, and the symptom translation module 360 may support AI algorithm integrated process and/or analysis in an effort to provide more accurate and convenient automated healthcare service to the patient. Some exemplary processes are disclosed in details with reference to FIGS. 4-8.

One of ordinary skill in the art shall understand that various processes, other than those disclosed in FIGS. 4-8, may also be implemented with the automated healthcare system. Those processes may comprise patient user registration/authentication, reference information retrieval via MICA, medical information translation for gathered medical information, automated diagnosis, and/or self-measurement.

FIG. 4 is an exemplary flow diagram illustrating a process of doctor authentication according to various embodiments of the present application. One of ordinary skill in the art shall understand that the registration and authentication process may be done locally or remotely by the system according to at least one authentication protocol after receiving input from the doctor.

At step 405, a doctor inputs information via API 310 in the patient apparatus 160. The API comprises instructions to guide the doctor to input information for authentication. The input information may comprise name, social security, address, contract information, username, password, etc. In some embodiments, the input information may also include biometric information, such as facial photo, fingerprint or IRIS, etc., which may be collected and used for doctor recognition/authentication. In embodiments, the facial photo or fingerprint of the patient is collected with the doctor apparatus (such as a smart phone) conveniently. The IRIS may be scanned using an IRIS scanner, which may be a standalone apparatus or an attachment to the doctor apparatus.

At step 410, the input information from the doctor is verified for authentication. The input information is compared to the established healthcare provider record, which may be stored within the patient/doctor management server 130. In embodiments, input information from the doctor is transmitted via secure link through the network 125 to the patient/doctor management server 130 for verification.

In step 415, once the authentication passed, the doctor is paired to a patient (or the doctor apparatus is paired to the patient apparatus) based on a pairing process. The pairing process may be a pre-determined process or a dynamically determined process. For example, the pairing process may be based on appointment, patient location, patient symptom, doctor's specialty, doctor's availability, cost standard, etc. In embodiments, the doctor 165 and the patient 110 (or the patient apparatus 120 and the doctor apparatus 160) are paired before the automated diagnostic started. In embodiments, the doctor 165 and the patient 110 (or the patient apparatus 120 and the doctor apparatus 160) are paired after an automated diagnostic module presents one or more diagnostic results and the pairing is dependent on the presented diagnostic results. The pairing process may be initiated by the patient or by the automated healthcare system. The pairing process may or may not need a confirmation from the doctor or a system administration staff who supervises appointment and pairing process.

In step 420, after paired to a patient, the doctor starts to receive patient information via the doctor apparatus. The patient information may comprise symptom input by the patient, retrieved electronic health record (EHR) of the patient, retrieved reference information from external resource(s) 355 via the MICA 350. In embodiments, the external resource 355 may be external an EHR database maintained by other healthcare providers, or medical insurance company. The external resource may also be a medical knowledge database, such as Medline, a bibliographic database of life sciences and biomedical information including bibliographic information for articles from academic journals covering medicine, nursing, pharmacy, dentistry, veterinary medicine, and health care. The reference information may be retrieved via a third party search engine, such a Google, OmniMedicalSearch, MedNets, Hardin MD, Welch Medical Library, PDR.net, ClinicalTrials.gov, Intute, Healthline, PubMed, MedConnect, Entrez, eMedicine, MedBioWorld, Electronic Orange Book, PubGene, MDLinx, Medscape, etc. For example, PubMed is a search engine accessing primarily the MEDLINE database of references and abstracts on life sciences and biomedical topics. PubMed is maintained as part of the Entrez system of information retrieval by the United States National Library of Medicine (NLM) at the National Institutes of Health. In embodiments, the reference information may be retrieved via public health institute or organization, such as Centers for Disease Control and Prevention (CDC), World Health Organization (WHO), International Federation of Red Cross and Red Crescent Societies.

In embodiments, the reference information may be retrieved from non-medical external sources, such as the patient's social medial activities. These information may be very important in help diagnose possible diseases. For example, after a patient inputs symptom, the MICA found from the patient's social media posts that the patient recently travelled to a country and also found from a report issued by CDC that there was a pandemic spreading across the country. The MICA further found that the symptom reported by the patient is highly related to the pandemic according to a recently publish medical journal article. All above information may be extremely helpful in diagnostic possible disease of the patient. Without the information from the patient's social media, it might take much longer time or more efforts for the automatic healthcare system to identify the disease.

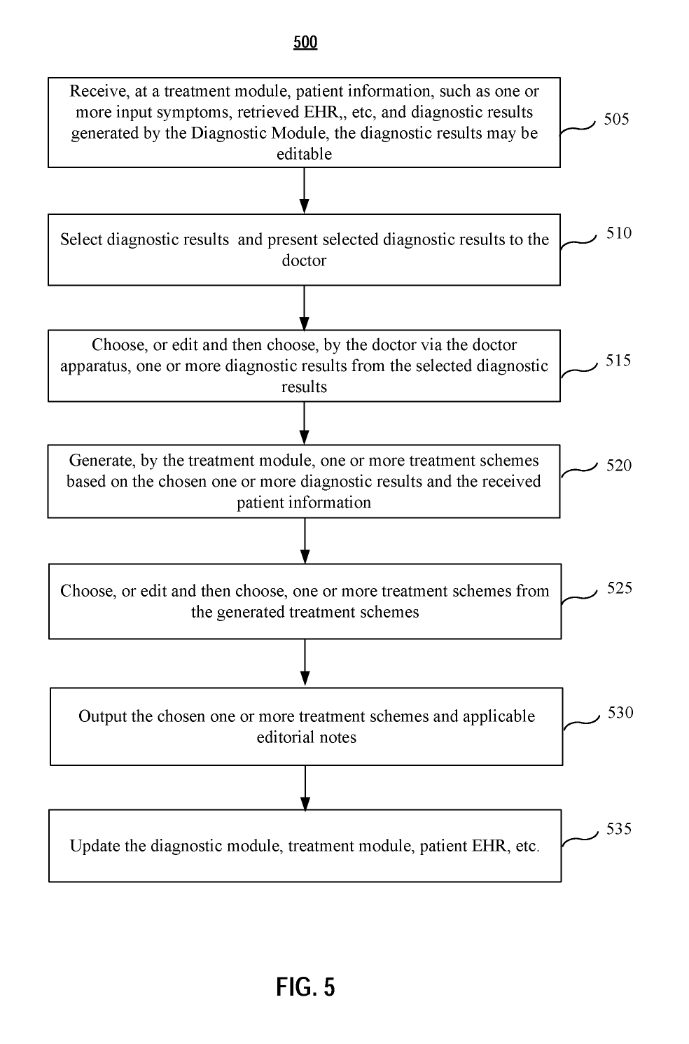

FIG. 5 is an exemplary flow diagram illustrating a process for diagnostic and treatment according to various embodiments of the present application. At step 505, patient information and diagnostic results generated by a diagnostic module 330 based on the patient information are received at a treatment module 340. The patient information may comprise one or more symptom inputs, electronic health record (EHR) of the patient, or retrieved reference information related to the patient or the symptom inputs. In embodiments, the diagnostic module 330 and the treatment module 340 may be separated modules operated in different devices. Alternatively, the diagnostic module 330 and the treatment module 340 may be integrated together as a single module operated in a computing apparatus. In embodiments, the diagnostic results generated by a diagnostic module 330 comprises a plurality of diagnostic results with each diagnostic result associating with a projected possibility based on a diagnosis analysis using the symptom inputs, patient EHR, and retrieved reference information related to the patient or the symptom inputs. The diagnostic results generated by a diagnostic module 330 may be a disease, an illness, an infection, a syndrome, a disorder, or a combination thereof. In embodiments, the diagnostic results may be editable by the doctor or other healthcare providers.

In embodiments, the diagnostic module and/or the treatment module may comprise artificial intelligence (AI) integrated algorithm and/or machine learning algorithms, such as recurrent neural network (RNN) or deep neural network (DNN), etc., for diagnosis and treatment analysis. In embodiments, the analysis process may comprise procedures of preprocessing, segmentation, region of interest analysis, evaluation, and classification, etc. In embodiments, the diagnostic engine may be pre-trained using known medical database before it is deployed online to improve the performance and efficiency.

At step 510, the treatment module selects one or more diagnosis results from the generated diagnostic results by the diagnostic module and presents the selected diagnosis results to the doctor. In embodiments, the selection process is based on the projected possibility of each diagnosis result and only the top N diagnosis results are selected. N is an integer number, such as 3, 5, 10, etc. The selected diagnosis results may be presented on the doctor apparatus in an order of the projected possibility. In embodiments, the treatment results may be editable as desired.

At step 515, the doctor chooses, or edits and then chooses, one or more diagnosis results from the presented diagnosis results as the final diagnosis results. The choice may be done via the doctor interface 310 and transmitted to the treatment module. In embodiments, the doctor may add some notes to the chosen diagnosis results. The notes may be a further explanation, a description that was not included or omitted by the diagnosis module. In embodiments, the doctor may even delete a result from a group of diagnosis results. The deleted result may be an unrelated result. The further editing capability provide enhanced robustness of the diagnosis process.

At step 520, the treatment module generates one or more treatment schemes based on the chosen one or more diagnostic results, the received patient information including symptom inputs, electronic health record (EHR) of the patient, and retrieved reference information. The generated one or more treatment schemes are also presented to the doctor for review. The treatment schemes may be a drug prescription, a physical therapy, a drug injection, a surgical operation, a physical exercise, a chiropractic treatment, an alternative medicine such as acupuncture or hypnosis, or a combination thereof.

In embodiments, each generated treatment scheme is associated with a projected possibility. The projected possibility is estimated based on a treatment analysis using the symptom inputs, patient EHR, retrieved reference information, the chosen one or more diagnostic results, as well as treatment reference information related to similar symptoms, etc. The treatment reference information may be retrieved from external resources via the MICA, and may comprise treatment schemes chosen by other healthcare providers with regards to similar symptoms, potential adverse effect associated to each treatment scheme, efficacy for the treatment scheme, cost for the treatment scheme, etc.

At step 525, the doctor chooses, or edits and then chooses, one or more treatment schemes from the generated treatment schemes as the final treatment schemes. The choice may be done via the doctor interface 310 and transmitted to the treatment module. In embodiments, the doctor may add some notes to the chosen treatment scheme. The notes may be a further explanation, such as duration of the treatment, cautions during the treatment, etc. In embodiments, the doctor may even delete a treatment schemes from a group of diagnosis results that were grouped together by the treatment module. The deleted result may be an unnecessary treatment scheme, or a scheme may have conflict from other schemes, or a scheme that may have too much side effects to the patient, etc. The further editing capability provides enhanced robustness of the treatment process.

At step 530, the chosen one or more treatment schemes, and applicable editorial notes, are output or transmitted to corresponding receptions, such as a drug store, a therapy facility, a lab, a nurse, etc., for implementation of the treatment schemes. The applicable editorial notes are referred as the editing at steps 510, 515, and/or 525.

At step 535, the choices of the doctor, including the choice of diagnostic results and treatment schemes, are used to update the diagnostic module, treatment module, patient EHR, etc. In embodiments, the choice of diagnostic results by the doctor is transmitted back to the diagnosis module before (or simultaneously with) the treatment scheme generation step 520.

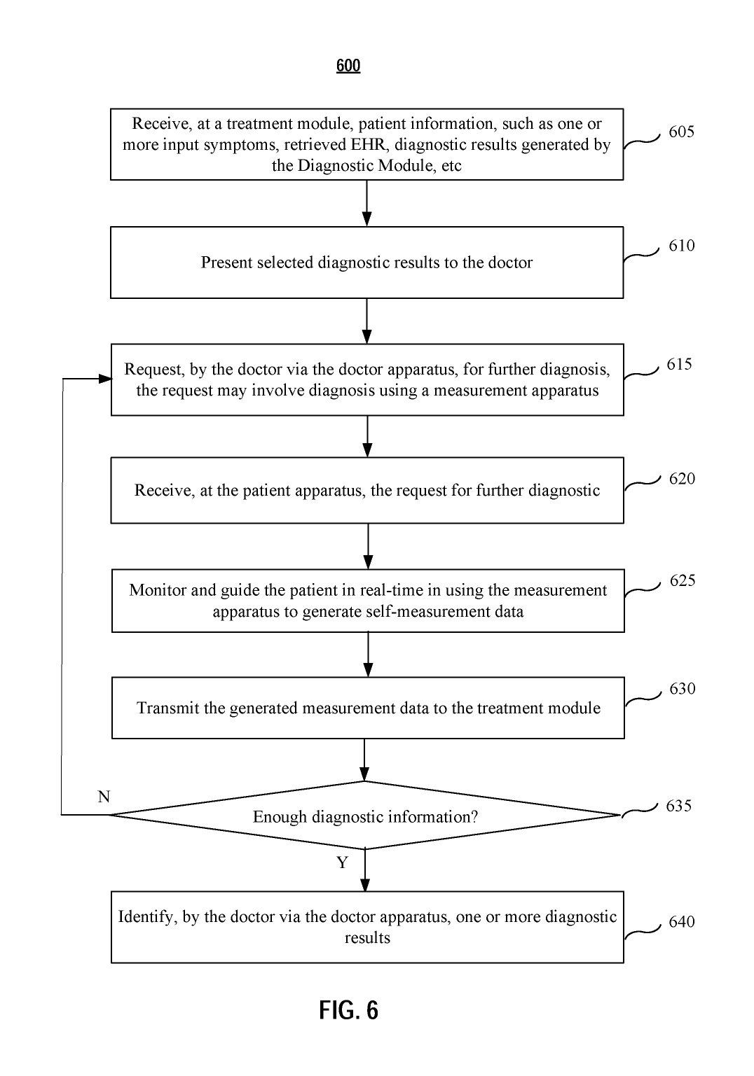

In embodiments, upon the presented diagnostic results, the doctor may feel that the some further diagnosis may be needed to confirm. FIG. 6 shows exemplary flow diagram illustrating a process for diagnostic and treatment under such scenario.

At step 605, patient information and diagnostic results generated by a diagnostic module based on the patient information are received at a treatment module. At step 610, the treatment module selects one or more diagnosis results from the generated diagnostic results by the diagnostic module and presents the selected diagnosis results to the doctor. The aforementioned remarks regarding step 505 and step 510 may also be applicable to steps 605 and step 610.

At step 615, after reviewing all the information received, the doctor may send a request via the doctor apparatus for further diagnosis. In embodiments, the further diagnosis request may involve diagnostics using a measurement apparatus, such as a self-measurement apparatus.

In embodiments, the self-measurement apparatus may be a heart rate sensors, otoscopes, a digital stethoscopes, an in-ear thermometers, a blood oxygen sensor, a high-definition camera, a spirometers, a blood pressure meter, a respiration sensor, a skin resistance sensor, a glucometer, an ultrasound devices, an electrocardiographic sensor, a body fluid sample collector, a weight scale, or any devices known in the art that may be self-operated in performing medical diagnosis. In embodiments, patient characteristics and vital signs data may be received from and/or compared against wearable or implantable monitoring devices that gather sample data, e.g., a fitness device that monitors physical activity. In embodiments, the self-measurement apparatus may be integrated within the patient apparatus or a stand-alone apparatus coupled to the patient apparatus. The self-measurement apparatus may contact a patient's body via patches or electrodes for good physical or electrical contact, or may perform contact-less measurements some distance away from the patient's body.

At step 620, the further diagnosis request is received at the patient apparatus 120 via a secure link through the network 125 and may be adaptively presented via the API 360 for the patient to respond. In embodiments, the way to present the request is based at least on patient medical history information. The questions may be adaptively presented in a text (with a specific size, font, color, etc.), audio, video, or a combination thereof. For example, if the patient medical history information shows that the patient had eye disease before, the further diagnosis request may be presented in an audio format, beside a text format. If the patient medical history information shows that the patient had hearing loss, the further diagnosis request may only be presented in a text format. If the patient medical history information shows that the patient is color blind, the further diagnosis request may be presented in a color that the patient is able to recognize.

In embodiments, when the further diagnosis request is presented to the patient, usage instruction of the self-measurement apparatus is also presented to the patient via the API 360. The instruction may comprise location of the apparatus, operation procedures, etc. The instruction may be presented in a text, audio, video, or an interactive stream format.

At step 625, the patient is guided and monitored in real-time in using the measurement apparatus (such as a self-measurement apparatus) to generate measurement data. The guidance and monitoring may be enabled by using a camera, an accelerometer, a multi-axis gyroscope, a pressure sensor, and/or a magnetometer to provide position, motion, pressure, orientation of the self-measurement apparatus based on patient interaction. In embodiments, a virtual reality (VR) algorithm is implemented for the guidance and monitoring. In embodiments, once the self-measurement apparatus is ready for measurement, a notice may be presented on the patient apparatus. The notice may be an icon, a voice message, a vibrating signal, or a combination thereof. Afterwards, the self-measurement apparatus begins the measurement and self-measurement data are generated. The data may be a digital image, a measurement reading, a video, etc.

In embodiments, the self-measurement process comprises a preliminary trustability check process. In embodiment, the generated self-measurement data is compared against a pre-determined range for trustability check. For example, a measured heartbeat rate should be within a reasonable range, such as between 40 and 120 beats per minute. A measured rate lower than 30 or higher than 200 may indicate a false measurement. If the preliminary trustability check passed, the process goes to step 630, in which the generated self-measurement data are transmitted to the treatment module (or the computing apparatus, the doctor apparatus). Otherwise, the self-measurement process repeats for a new round of measurement guidance and monitoring for another self-measurement trial.

At step 635, the generated self-measurement data are presented for the doctor to determine whether available diagnosis information is enough for a decision. If yes, the process goes to step 640. If not, the step goes back to 615 for another round of self-measurement with another self-measurement apparatus.

At step 640, the doctor identifies, via the doctor apparatus, one or more diagnostic results based on all available information. The identified one or more diagnostic results may be chosen from the diagnostic results generated by the diagnosis module, or newly identified based at least on the generated self-measurement data. In embodiments, after the doctor identifies one or more diagnostic results, the process may go to step 520 (as shown in FIG. 5) to continue the treatment process.

One of ordinary skill in the art may understand that the aforementioned process in step 615 to step 630 involving a self-measurement apparatus is also applicable to the situation of requesting more symptom input or confirming whether any other symptoms exist, instead of using a self-measurement apparatus.

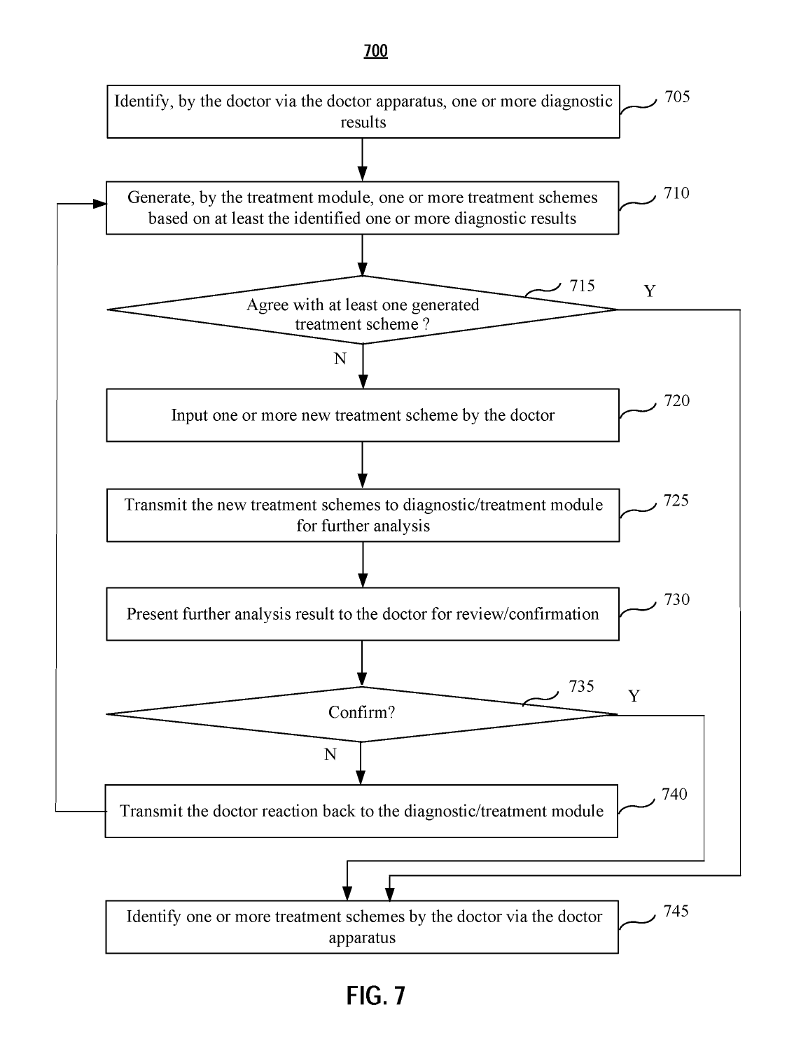

In embodiments, the doctor may not agree with any treatment schemes presented by the treatment module and may input a new treatment scheme for various reasons. FIG. 7 shows an exemplary flow diagram illustrating such a process for diagnostic and treatment according to various embodiments of the present application.

At step 705, the doctor identifies, via the doctor apparatus, one or more diagnostic results based on all available information.

At step 710, the treatment module generates one or more treatment schemes based on the chosen one or more diagnostic results, the received patient information including symptom inputs, electronic health record (EHR) of the patient, and retrieved reference information. The generated one or more treatment schemes are presented to the doctor for review. The treatment schemes may be a drug prescription, a physical therapy, a drug injection, a surgical operation, a physical exercise, a chiropractic treatment, an alternative medicine such as acupuncture or hypnosis, or a combination thereof. In embodiments, each generated treatment scheme is associated with a projected possibility. The projected possibility is estimated based on a treatment analysis using the symptom inputs, patient EHR, retrieved reference information, the chosen one or more diagnostic results, as well as treatment reference information related to similar symptoms, etc. The treatment reference information may be retrieved from external resources via the MICA, and may comprise treatment schemes chosen by other healthcare providers with regards to similar symptoms, potential adverse effect associated to each treatment scheme, efficacy for the treatment scheme, cost for the treatment scheme, etc.

In embodiments, the treatment module may comprise AI integrated algorithm and/or machine learning algorithms, such as recurrent neural network (RNN) or deep neural network (DNN), etc., for treatment analysis. In embodiments, the analysis process may comprise procedures of preprocessing, segmentation, region of interest analysis, evaluation, and classification, etc. In embodiments, the treatment module may be pre-trained using known medical database before it is deployed online to improve the performance and efficiency.

At step 715, a decision is made by the doctor to determine whether at least one generated treatment scheme is agreeable. If yes, the process goes to step 745, in which the doctor identifies one or more treatment schemes via the doctor apparatus and then goes to step 530 to continue.

If none of the generated treatment scheme is agreeable, the process goes to step 720, in which the doctor may manually input one or more new treatment schemes.

In embodiments, at step 725, the one or more new treatment schemes are transmitted to the diagnosis module and/or the treatment module for further analysis (such as a risk analysis). The purpose of the risk analysis may be related to malpractice prevention. In embodiments, the risk analysis may use AI integrated algorithm and/or machine learning algorithms, such as recurrent neural network (RNN) or deep neural network (DNN), etc., to collect information, such as medical examples associated with the one or more new treatment schemes, potential adverse effects of the one or more new treatment schemes, medicines that are incompatible with the one or more new treatment schemes, etc. The collected information may be sorted, prioritized during the risk analysis.

At step 730, the further analysis result is presented to doctor via the API 310 for review/confirmation. At step 735, upon reviewing the further analysis result, the doctor makes a decision whether to confirm the one or more new treatment schemes. If yes, the process goes to step 745, in which the doctor identifies one or more treatment schemes (the one or more new treatment schemes) via the doctor apparatus. In embodiments, the process may then goes to step 535 and step 540 to output the treatment schemes for implementation and to update the diagnosis module, treatment module and the patient EHR.

If the doctor does not confirm the one or more new treatment schemes after review the presented further analysis result, the process goes to step 740, in which the doctor's reaction is transmitted back to the diagnosis/treatment module for update. Afterwards, the process goes back to step 710 for a new round of treatment scheme generation.

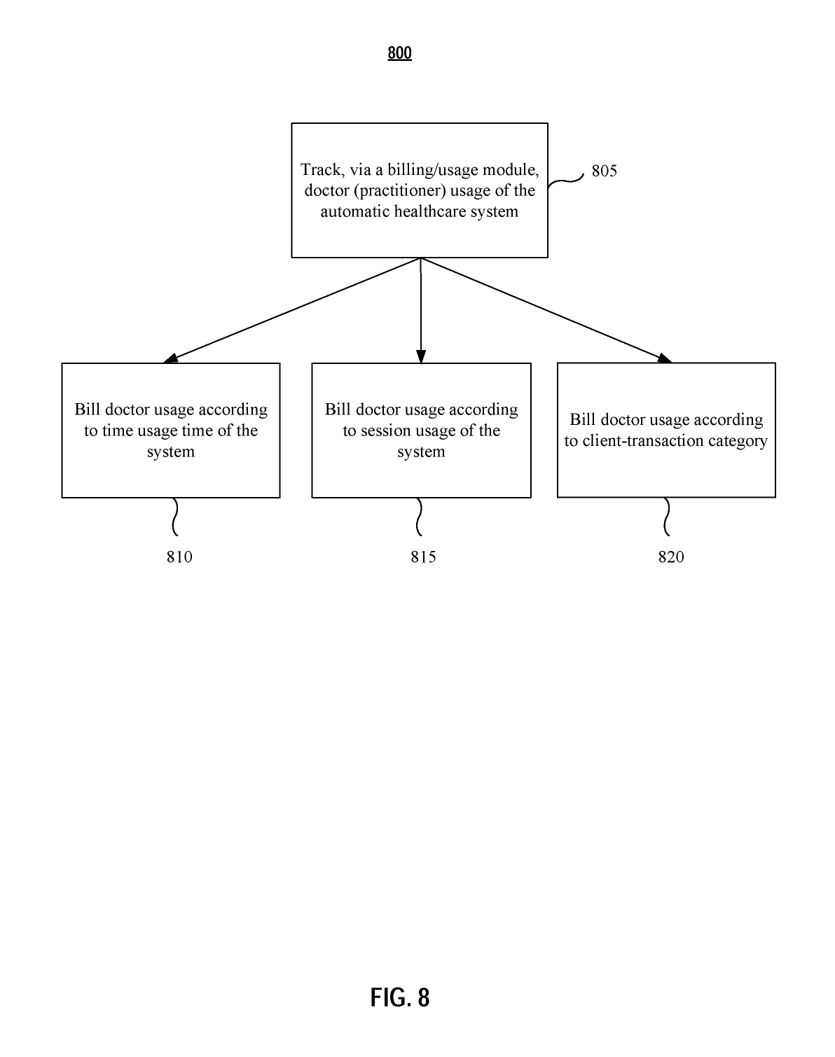

In embodiments, the automated diagnosis and treatment process also enables automated billing process. FIG. 8 is an exemplary flow diagram illustrating a process of billing according to various embodiments of the present application.

At step 805, doctor usage of the automatic healthcare system is tracked by a billing (or usage) module. The usage module may be incorporated within the doctor apparatus and may operate as a background app. The usage module may incorporate one or more times for time usage tracking, one or more network traffic meters for network traffic monitoring and gathering statistics in order to control and analyze.

Based on the tracked usage information, various billing methods or steps may be implemented via a billing module. The billing module couples to the usage module to receive track usage information, such as usage time, usage traffic, categories of the usage traffic, etc.

The billing module and the usage module may be integrated into a single module, which may be installed within the doctor apparatus. Alternatively, the usage module is installed within the doctor apparatus, while the billing module may be incorporated within the doctor/patient management server, or inside a third party apparatus, such as a server of the automated healthcare system developer. The generated billing invoice for the doctor may be used to collect payment from the doctor, and also used as a basis to calculate dues to be collected from the patient, or healthcare insurance companies.

In embodiments, in step 810, doctor usage is billed by a billing module according to the actual use time of the system. Alternatively, in step 815, doctor usage is billed by a billing module according to session usage of the system. The session usage may be counted based on the number of patients that the doctor serves, etc. Alternatively, in step 820, doctor usage is billed by a billing module according to client-transaction category. For example, the billing rate for a complex symptom diagnosis and treatment involving more analysis or searching in specialty database may have a higher billing rate compared to a simple symptom. In embodiments, the actual billing method may be a combination of steps 810, 815 and 820.

In embodiments, if a doctor provides a diagnosis and/or treatment scheme not presented by the automated healthcare system in identifying or treating a rare disease, the billing module may offer credits to the doctor for the discovery. Such a discovery may be very helpful in updated knowledge database for diagnosis and treatment modules.

Although FIG. 4-FIG. 8 show steps in individual process of the automatic healthcare service for diagnosis and treatment, one skilled in the art shall recognize that: (1) certain steps may optionally be performed; (2) steps may not be limited to the specific order set forth herein; (3) certain steps may be performed in different orders; (4) certain steps in FIG. 4-FIG. 8 may be done concurrently; and (4) certain steps in FIG. 4-FIG. 8 may be cross-linked.

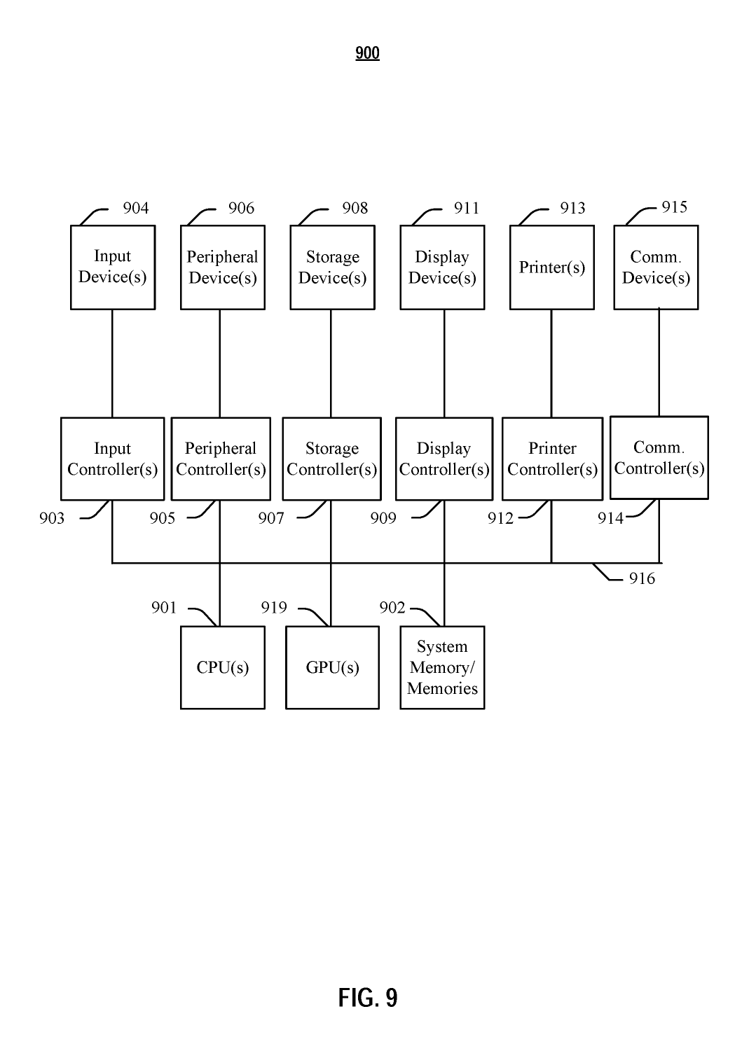

In embodiments, aspects of the present patent document may be directed to, may include, or may be implemented on one or more computing systems. A computing system may include any instrumentality or aggregate of instrumentalities operable to compute, calculate, determine, classify, process, transmit, receive, retrieve, originate, route, switch, store, display, communicate, manifest, detect, record, reproduce, handle, or utilize any form of information, intelligence, or data. An exemplary system that may be used to complement one or more aspects of the present disclosure is described with referent to FIG. 9. Each of the aforementioned patient apparatus, doctor apparatus, computing apparatus, patient management server, usage/billing server, and/or self-measurement apparatus may comprise one or more components in the system 900. It will be understood that the functionalities shown for system 900 may operate to support various embodiments of a computing system—although it shall be understood that a computing system may be differently configured and include different components, including having fewer or more components as depicted in FIG. 9.

As illustrated in FIG. 9, the computing system 900 includes one or more central processing units (CPU) 901 that provides computing resources and controls the computer. CPU 901 may be implemented with a microprocessor or the like, and may also include one or more graphics processing units (GPU) 919 and/or a floating-point coprocessor for mathematical computations. System 900 may also include a system memory 902, which may be in the form of random-access memory (RAM), read-only memory (ROM), or both.

A number of controllers and peripheral devices may also be provided, as shown in FIG. 9. An input controller 903 represents an interface to various input device(s) 904, such as a keyboard, mouse, touchscreen, and/or stylus. The computing system 900 may also include a storage controller 907 for interfacing with one or more storage devices 908 each of which includes a storage medium such as magnetic tape or disk, or an optical medium that might be used to record programs of instructions for operating systems, utilities, and applications, which may include embodiments of programs that implement various aspects of the present invention. Storage device(s) 908 may also be used to store processed data or data to be processed in accordance with the invention. The system 900 may also include a display controller 909 for providing an interface to a display device 911, which may be a cathode ray tube (CRT), a thin film transistor (TFT) display, organic light-emitting diode, electroluminescent panel, plasma panel, or other type of display. The computing system 900 may also include one or more peripheral controllers or interfaces 905 for one or more peripherals 906. Examples of peripherals may include one or more printers, scanners, input devices, output devices, sensors, and the like. A communications controller 914 may interface with one or more communication devices 915, which enables the system 900 to connect to remote devices through any of a variety of networks including the Internet, a cloud resource (e.g., an Ethernet cloud, an Fiber Channel over Ethernet (FCoE)/Data Center Bridging (DCB) cloud, etc.), a local area network (LAN), a wide area network (WAN), a storage area network (SAN) or through any suitable electromagnetic carrier signals including infrared signals.

In the illustrated system, all major system components may connect to a bus 916, which may represent more than one physical bus. However, various system components may or may not be in physical proximity to one another. For example, input data and/or output data may be remotely transmitted from one physical location to another. In addition, programs that implement various aspects of the invention may be accessed from a remote location (e.g., a server) over a network. Such data and/or programs may be conveyed through any of a variety of machine-readable medium including, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices.

Aspects of the present invention may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present invention may further relate to computer products with a non-transitory, tangible computer-readable medium that have computer code thereon for performing various computer-implemented operations. The media and computer code may be those specially designed and constructed for the purposes of the present invention, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present invention may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the present invention. One skilled in the art will also recognize that a number of the elements described above may be physically and/or functionally separated into sub-modules or combined together.

It will be appreciated to those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

The foregoing description of the present application has been described for purposes of clarity and understanding. It is not intended to limit the present application to the precise form disclosed. Various modifications may be possible within the scope and equivalence of the appended claims.

It will be appreciated to those skilled in the art that the preceding examples and embodiments are exemplary and not limiting to the scope of the present application. It is intended that all permutations, enhancements, equivalents, combinations, and improvements thereto that are apparent to those skilled in the art upon a reading of the specification and a study of the drawings are included within the true spirit and scope of the present application.

It shall also be noted that elements of the claims, below, may be arranged differently including having multiple dependencies, configurations, and combinations. For example, in embodiments, the subject matter of various claims may be combined with other claims.

We claim:

1. A method for automated healthcare service, the method comprising:
receiving one or more of diagnosis results generated by a diagnosis module, the generated one or more diagnosis results being related to one or more input symptoms from a patient via a patient interface;
selecting one or more diagnosis results among the generated one or more diagnosis results;
choosing or editing and then choosing at least one diagnosis result from the selected one or more diagnosis result;
generating, using a treatment module, one or more treatment schemes based on the chosen diagnosis result;
choosing or editing and choosing one or more treatment schemes from the generated one or more treatment schemes; and
outputting the chosen one or more treatment schemes for implementation.

2. The method of claim 1, wherein each generated diagnostic result is associated with a projected possibility based on a diagnosis analysis using the input symptoms, retrieved patient electronic health record, and retrieved reference information related to the patient or the input symptoms.

3. The method of claim 2, wherein selecting one or more diagnosis results is based on the projected possibility of each generated diagnosis result.

4. The method of claim 3, wherein only the top N diagnosis results are selected, N is an integer number larger than 1.

5. The method of claim 1, wherein the treatment module implements a treatment analysis using the input symptoms, retrieved patient electronic health record, the chosen diagnosis result, or treatment reference information related to symptoms similar to the chosen diagnosis result.

6. The method of claim 5, wherein the retrieved treatment reference information comprise treatment schemes chosen by other healthcare providers, potential adverse effect associated to each treatment scheme, efficacy for the treatment scheme, or cost for the treatment scheme.

7. The method of claim 1 further comprises:
updating the diagnosis module using the one or more chosen diagnosis results; and
updating the treatment module using the one or more chosen treatment schemes.

8. A system for automated healthcare service, the system comprising:
a patient apparatus comprising a patient interface to receive patient input symptoms;
a diagnostic module coupled to receive the patient input symptoms and associated electronic health record related to the patient, the diagnostic module implements a diagnosis analysis to generate one or more diagnosis results, among the generated one or more diagnosis results, one or more diagnosis results are selected and transmitted;
a practitioner apparatus coupled to the patient apparatus and the diagnostic module, the healthcare apparatus receives the selected one or more diagnosis results, the patient input symptoms and associated electronic health record related to the patient, the practitioner apparatus comprising a practitioner interface to receive input from a practitioner to identify one or more diagnostic results based at least on patient input symptoms, associated electronic health record and the selected one or more diagnosis results; and
a treatment module coupled to receive the identified one or more diagnostic results, the treatment module generates one or more treatment schemes based on at least the identified one or more diagnostic results, the generates one or more treatment schemes are transmitted to the practitioner apparatus for the practitioner to choose, or edit and then choose, one or more treatment schemes among the generates treatment schemes.

9. The system of claim 8, wherein the identified one or more diagnostic results are chosen among the selected one or more diagnosis results transmitted from the diagnosis module.

10. The system of claim 8, wherein the identified one or more diagnostic results are newly identified diagnostic results, which are not among the selected one or more diagnosis results transmitted from the diagnosis module.

11. The system of claim 10, wherein the newly identified diagnostic results are identified based on further diagnosis result in response to a request for further diagnosis, the request being transmitted from the practitioner apparatus to the patient apparatus.

12. The system of claim 11, wherein the request for additional patient input is a request for the patient to use a measurement apparatus to obtain a measurement diagnostic result.

13. The system of claim 8 further comprising a usage module to track practitioner usage of the automated healthcare service system.

14. The system of claim 13 further comprising a billing module coupled to the usage module and generating bills according to time usage, session usage, client-transaction category, or a combination thereof.

15. A method for automated diagnosis and treatment, the method comprising:
receiving symptom input from a patient via a patient interface within the patient apparatus;
generating, using a diagnosis module, one or more of diagnosis results related to one or more input symptoms;
identifying, by a healthcare practitioner via a practitioner apparatus, one or more diagnosis results among the generated one or more diagnosis results;
generating, using a treatment module, one or more treatment schemes related to one or more identified diagnosis results;
presenting the generated one or more treatment schemes to the healthcare practitioner; and
in response to one or more treatment schemes are chosen or chosen with editing among the generated one or more treatment schemes by the healthcare practitioner, outputting the chosen one or more treatment schemes;
in response to none of the generated one or more treatment schemes is chosen by the healthcare practitioner, inputting one or more new treatment schemes by the healthcare practitioner.

16. The method of claim 15, wherein the one or more new treatment schemes are transmitted back to the treatment module for further analysis.

17. The method of claim 16, wherein the further analysis is a risk analysis may be related to malpractice prevention.

18. The method of claim 16, wherein the further analysis is transmitted back to the practitioner apparatus for the healthcare practitioner to decide whether to confirm the one or more new treatment schemes.

19. The method of claim 18, wherein
in response to the one or more new treatment schemes are confirmed, outputting the one or more new treatment schemes as identified treatment schemes in response to the symptom input from the patient;
in response to the one or more new treatment schemes are not confirmed, transmitting the not confirming response back to the treatment module for a new round of treatment scheme generation.

20. The method of claim 19 further comprising:
in response to the one or more new treatment schemes are confirmed, updating the treatment module with the identified treatment schemes.
